SEQUENCE LISTING (12 sequences)

SEQ ID NO: 1 - DNA, 20 bp, Artificial Sequence
  Description of Artificial Sequencebeta-actin semiquantitative RT-PCR Forward primer
cctaaggccaaccgtgaaaa

SEQ ID NO: 2 - DNA, 21 bp, Artificial Sequence
  Description of Artificial Sequencebeta-actin semiquantitative RT-PCR Reverse primer
gaggcatacagggacagcaca

SEQ ID NO: 3 - DNA, 19 bp, Artificial Sequence
  Description of Artificial Sequencemdr-1a semiquantitative RT-PCR Forward primer
agctggagagatcctcacc

SEQ ID NO: 4 - DNA, 20 bp, Artificial Sequence
  Description of Artificial Sequencemdr-1a semiquantitative RT-PCR Reverse primer
ctgtagctgtcaatctcggg

SEQ ID NO: 5 - DNA, 19 bp, Artificial Sequence
  Description of Artificial Sequencemdr-1b semiquantitative RT-PCR Forward primer
agccggagagatcctcacc

SEQ ID NO: 6 - DNA, 20 bp, Artificial Sequence
  Description of Artificial Sequencemdr-1b semiquantitative RT-PCR Reverse primer
ctgtagctgtcaatctcagg

SEQ ID NO: 7 - DNA, 19 bp, Artificial Sequence
  Description of Artificial Sequencemdr-2 semiquantitative RT-PCR Forward primer
agctggagagatcctcacc

SEQ ID NO: 8 - DNA, 20 bp, Artificial Sequence
  Description of Artificial Sequencemdr-2 semiquantitative RT-PCR Reverse primer
ctgtagctgtcaatcagagg

SEQ ID NO: 9 - DNA, 20 bp, Artificial Sequence
  Description of Artificial Sequencemrp-1 semiquantitative RT-PCR Forward primer
ggcgctgtctatcgtaaggc

SEQ ID NO: 10 - DNA, 19 bp, Artificial Sequence
  Description of Artificial Sequencemrp-1 semiquantitative RT-PCR Reverse primer
gacctccgctcaatgctgt

SEQ ID NO: 11 - DNA, 24 bp, Artificial Sequence
  Description of Artificial Sequencemrp-2 semiquantitative RT-PCR Forward primer
tgcctgtcctataactcacggatt

SEQ ID NO: 12 - DNA, 26 bp, Artificial Sequence
  Description of Artificial Sequencemrp-2 semiquantitative RT-PCR Reverse primer
agcaaatgttattgtttgtaggtccg

(19) United States
(12) Patent Application Publication
(10) Pub. No.: US 2006/0057657 A1
(43) Pub. Date: Mar. 16, 2006

(54) Stem cell selection and differentiation

(75) Inventors: Manfred Baetscher, Portland, OR (US); Jenefer Dekoning, Vancouver, WA (US); Anne Bower, Portland, OR (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Oregon Health & Science University Technology & Research Collaborations, Portland, OR (US)

(21) Appl. No.: 10/507,384

(22) PCT Filed: Mar. 12, 2003
(86) PCT No.: PCT/US03/08259

§ 371 Date: Aug. 12, 2005

Related U.S. Application Data
(60) Provisional application No. 60/365,022, filed on Mar. 12, 2002.

(51) Int. Cl. C12Q 1/62; C12N 5/08

(52) U.S. Cl. 435/10; 435/366

(57) ABSTRACT

Isolated mammalian stem cells sustainable in culture under glycolytic conditions and which maintain the potential to differentiate are provided. Further encompassed by the invention are functionally distinct subpopulations of stem cells with increased differentiation permissiveness.

This application claims the benefit of U.S. Provisional Application No. 60/365,022, filed Mar. 12, 2002.

### BACKGROUND OF THE INVENTION

a) Field of the Invention

This invention relates to stem cells, particularly to sustainable stem cell lines and methods for identifying a subpopulation of the stem cells with a high potential for differentiation.

b) Description of Related Art

In general, stem cells are undifferentiated cells which can give rise to a succession of mature functional cells. For example, a hematopoietic stem cell may give rise to any of the different types of terminally differentiated blood cells. Embryonic stem (ES) cells are derived from the embryo and are pluripotent, thus possessing the capability of developing into any organ or tissue type or, at least potentially, into a complete embryo.

One of the seminal achievements of mammalian embryology of the last decade has been the routine insertion of specific genes into the mouse genome through the use of mouse ES cells. This genetic alteration has created a bridge between the in vitro manipulations of molecular biology and an understanding of gene function in the intact animal. Mouse ES cells are undifferentiated, pluripotent cells derived in vitro from preimplantation embryos. Functionally related to ES cells are embryonic germ (EG) cells which are derived from primordial germ cells or fetal germ cells. Mouse ES cells maintain an undifferentiated state through serial passages when cultured on fibroblast feeder layers in the presence of Leukemia Inhibitory Factor (LIF). If LIF is removed, in the absence of feeder layers, mouse ES cells will differentiate.

The ability of mouse ES cells to contribute to functional germ cells in chimeras provides a method for introducing site-specific mutations into mouse lines. With appropriate transfection and selection strategies, homologous recombination can be used to derive ES cell lines with planned alterations of specific genes. These genetically altered cells can be used to form chimeras with normal embryos and chimeric animals can be recovered. If the ES cells contribute to the germ line in the chimeric animal, then in the next generation a mouse line for the planned mutation is established. Because mouse ES cells have the potential to differentiate into any cell type in the body, mouse ES cells allow the in vitro study of the mechanisms controlling the differentiation of specific cells or tissues.

Stem cells have the ability to commit to either of two basic cell-fate decisions: self-renewal or differentiation. Some stem cells are pluripotent (embryo-derived stem (ES) cells, hematopoietic stem cells); others are unipotent (spermatogonia). Certain stem cell compartments, like the inner cell mass (ICM), the origin of ES cells in the blastocyst stage embryo, are transient; others, such as the hematopoietic stem cells in the bone marrow, persist in the adult organism.

Embryonic stem (ES) cells derived from the ICM of blastocysts can be maintained undifferentiated in culture indefinitely when grown in the presence of feeder cells or specific cytokines. In the absence of these reagents, they differentiate into embryoid bodies or they form embryonic carcinomas in immuno-compromised mice. Mouse ES cells can also be genetically manipulated and clonally selected; when re-injected into a host blastocyst stage embryo, they can differentiate into all somatic cell lineages and the germ line.

While ES cells can replicate indefinitely, their ability to differentiate decreases with the number of population doublings. This limitation has practical implications, as ES cells used for gene targeting experiments must invariably give rise to germ cells, and failure to do so frequently renders projects unsuccessful. Therefore, only early passage cells are desirable due to the ES cells propensity to drift. Understanding the molecular processes underlying this phenomenon would be advantageous. It might also help to explain why targeted mutagenesis has been unsuccessful in mammalian species other than mouse.

Recent advances in culturing human embryonic stem (ES) cells suggest that these cells are important for treatment of diseases which are due to cell loss or damage (Robertson, J. A. (2001) Nat. Rev. Genet. 2:74-78; Thomson et al (1998) Science 282:1145-1147; Tsai et al (2002) Dev. Cell 2:707-712). This has led to increased interest in the development of protocols for differentiation of stem cells, and it also spurred an interest in studies about regulatory mechanisms governing ES cells (Smith, A. G. (2001) Annu. Rev. Cell Dev. Biol. 17:435-462). Human ES cells derived from the ICM or from primordial germ cells share some of the characteristics of mouse ES cells, such as immortality, as shown by sustained telomerase activity and the formation of teratoma tumors in SCID mice. The most prominent properties specific to ES cells include self-renewal and pluripotency. However, human ES cells require feeder cells and cannot remain undifferentiated in the presence of cytokines such as Leukemia Inhibitory Factor (LIF). In fact, self-renewal depends on activation of STAT3 by tyrosine phosphorylation which is mediated by gp130 (Narazaki et al. (1994) Proc. Natl. Acad. Sci. USA 91:2285-2289; Yoshida et al. (1994) Mech. Dev. 45:163-171) upon binding of ligands such as LIF (Smith et al. (1988) Nature 336:688-690; Williams et al. (1988) Nature 336:684-687) or IL-6 (Ernst et al. (1994) Embo J. 13:1574-1584); Nichols et al. (1994) Exp. Cell Res. 215:237-239). Pluripotency of ES cells is at least in part dependent on the expression of a defined amount of the octamer-binding transcription factor, Oct 3/4 (Nichols et al. (1998) Cell 95:379-391). However, while Oct 3/4 is necessary to maintain the pluripotent state of ES cells, it does not restore pluripotency in differentiated cells. Generally, the growth rate of human ES cells is slower and they are much more difficult to maintain in vitro than mouse ES cells. Human ES cells are the focus of extensive research efforts, as they hold significant promise for future clinical treatments, including tissue regeneration.

Differentiation of ES cells is initiated upon removal of reagents supporting self-renewal when cells are cultured in the absence of a feeder layer or cytokines activating the gp130/STAT3 pathway. In vitro, this process is facilitated by exposing cells to retinoic acid which, after prolonged culture, results in the formation of embryoid bodies (Bain et al. (1995) Dev. Biol. 168:342-357; Dani et al. (1997) J. Cell Sci. 110:1279-1285). These amorphous bodies, including a number of different cell types, can be exposed to specific growth factors to induce proliferation and differentiation of selective cell lineages.

The differentiation potential of stem cells in vivo has been studied most extensively in the pluripotent hematopoietic stem cells (HSC) from the bone marrow (Weissman et al. (2001) Annu. Rev. Cell Dev. Biol. 17:387-403). The “true” HSC has the capacity to repopulate all hematopoietic lineages long term. This reconstitution in vivo is in concept comparable to ES cells colonizing all lineages including the germ line in a chimera. In bone marrow, isolation of HSC is dependent on their expression of cell surface antigens such as c-kit (+), Thy 1.1 (lo), Lin (−), and Scal (KTLS-HSC) (Christensen and Weissman (2001) Proc. Natl. Acad. Sci. USA 98:14541-14546), as well as the fluorescence signal following staining with the vital dye, Hoechst 33342 (Hoechst) (Goodell et al. (1996) J. Exp. Med. 183:1797-1806). Flow cytometry of HSCs using Hoechst reveals a characteristic side population (SP) that corresponds to a small population of cells with long term repopulating activity (Goodell et al., supra). The Hoechst SP is sensitive to inhibition with verapamil, cyclosporin A, and reserpine, a class of drugs that block members of the family of multidrug resistance (MDR) genes. These genes, which encode, among others, the drug efflux pump, permeability glycoprotein (P-gp), belong to the large family of ATP-binding cassette transporters, also termed ABC transporters (Gottesman and Ambudkar (2001) J. Bioenerg. Biomembr. 33:453-458). ABC transporters catalyze the energy dependent transport of chemical substances across membranes (Gottesman and Ambudkar, supra). They share a characteristic ATP-binding domain but otherwise are very diverse, possessing a wide range of substrate specificities, expression profiles, and subcellular locations. Mutations in ABC transporter genes often are associated with human disease (Gottesman and Ambudkar, supra), such as in cystic fibrosis which is caused by a mutated CFTR gene. ABC transporters have recently become markers for the phenotypic characterization of stem cells (Bunting, K. D., (2002) Stem Cells 20:11-20). For example, the Hoechst SP phenotype in HSC has been shown to be due to more than one ABC transporter activity (Uchida et al. (2002) Exp. Hematol. 30:862-869). In ES cells, possible candidate transporters are likely to be members of the MDR and multidrug resistance associated protein (MRP) gene families because of their ubiquitous expression pattern and broad substrate specificity. In addition, MDR-1, MRP-1, and ABCG2 have been implicated in multidrug resistance of virtually all types of tumor cell lines. Therefore, these transporters are the most likely candidates to be active in ES cells, which share many of the properties of tumor cells.

Energy metabolism and production of ATP are essential to all living organisms, including stem cells. Under normal oxygen conditions, cells produce most of their energy by oxidative phosphorylation (OXPHOS) in the mitochondria. Certain environmental conditions, such as hypoxia, cause cells to adapt by switching to glycolysis (Pasteur effect). This produces less ATP, but, in the presence of sufficient glucose, cells can meet their energy demands through glycolysis. This adaptation to glycolysis has been described most notably for tumors. The molecular mechanisms controlling energy metabolism and the switch between OXPHOS and glycolysis involve the hypoxia-inducible factors (HIF). HIF's are transcription factors of the basic helix-loop-helix DNA-binding protein family. They directly regulate a number of genes involved in glycolysis, erythropoiesis and vasculogenesis. While there may be a basic understanding of the role of stem cells in the development of multicellular organisms, there remains a need for further insights into stem cell functionality as well as for development of methods for influencing their differentiation and longevity.

### BRIEF SUMMARY OF THE INVENTION

A cell's energy metabolism depends on both glycolysis and oxidative phosphorylation (OXPHOS). Cultured stem cells, particularly embryonic stem (ES) cells, use either both glycolysis and OXPHOS or rely on glycolysis similar to cells exposed to hypoxic conditions. These cells preferably rely on glycolysis for energy metabolism, which is a property normally found in certain aggressive forms of tumor cells. Isolated stem cells sustainable in culture under glycolytic conditions and which maintain the potential to differentiate are provided. The stem cells may be unipotent or pluripotent. The stem cells are embryonic or somatic stem cells. The stem cells may be pluripotent cells from a preimplantation embryo or primordial germ cells. The stem cells may be hematopoietic, neuronal or mesenchymal stem cells. The isolated stem cells may stain with the mitochondrial marker JC-1 and emit a characteristic green fluorescent signal.

One aspect of the invention provides methods of isolating a pluripotent stem cell, comprising the steps of isolating a blastocyst, identifying those cells which rely upon glycolysis for survival, isolating a glycolytic cell from the inner cell mass of the blastocyst, and culturing the isolated glycolytic cell to obtain an isolated stem cell. Such cells are identified by staining with the mitochondrial marker JC-1. The invention also embodies the selection and isolation of potentially glycolytic cells from a batch of cultured stem cells. The cultured stem cells may engage in OXPHOS and glycolytic energy metabolism.

Another aspect of the invention provides for the injection of stained and JC-1 flow sorted cells into blastocyst stage embryos, followed by transfer of the injected embryos into pseudopregnant recipient foster mother mice. Offspring animals may be derived from JC-1 stained, flow sorted, blastocyst injected embryos. Such offspring are heavily chimeric as evidenced by coat pigmentation being almost entirely derived from the genotype of the stained and sorted stem cells. Cells that are stained, flow sorted and injected into blastocysts are also found to differentiate into germ cells of chimeric animals.

Further encompassed are chimeric animals produced from an isolated cell of this invention. In one aspect a chimeric animal may be produced by isolating a blastocyst, identifying those cells which rely upon glycolysis for survival, isolating the glycolytic cells from the inner cell mass of the blastocyst, transfecting a desired gene into the glycolytic cells, injecting the transfected cells into recipient blastocysts, implanting the transformed blastocysts into a host uterus, and nurturing the blastocysts to develop to term. In order to generate nerve or blood cells, ES cells may be differentiated in a concerted fashion and shifted to JC-1 green cells before initiating differentiation.

Also contemplated by the instant invention are subpopulations of ES cells that can be identified using the fluorescent dye, JC-1. The cells stained with JC-1, followed by fluorescence activated cell sorting (FACS) analysis, show two functionally distinct subpopulations of ES cells that differ in their ability to produce chimeras. These subpopulations are sensitive to inhibitors of multidrug resistance (MDR) targets. The inhibitors include, but are not limited to, verapamil, reserpine, and cyclosporine A. This sensitivity suggests the involvement of MDR targets, such as MDR-like dye efflux pumps. In fact, various MDR gene family members are expressed at greater levels in the drug-sensitive subpopulations, specifically mdr1a/1b and mrp-1. The MDR gene family members define the Hoechst 33342 side population (SP) in hematopoietic stem cells (HSC). A related SP is contemplated for ES cells. A comparison of the Hoechst 33342 and JC-1 profiles following double staining and flow cytometry shows that the MDR-inhibitor sensitive JC-1 subpopulation, which has increased differentiation permissiveness, identifies a subset of cells within the Hoechst SP showing a characteristic linear population. Hence, the invention identifies functionally distinct ES cell subpopulations, wherein some MDR genes may be used as markers to identify and manipulate the differentiation permissiveness of ES cells.

Another aspect of the invention provides for a method of switching embryonic stem cells between two subpopulations, comprising exposing a JC-1 green subpopulation to inhibitors of multidrug resistance genes; and overexpressing recombinant multidrug resistance genes in a JC-1 red subpopulation. The inhibitors include, but are not limited to, verapamil, reserpine and cyclosporine. The invention further provides for a method of changing a cell's ability to differentiate by switching subpopulations of cells. An embryonic stem cell which is differentiated by switching the subpopulations of cells is also provided.

### BRIEF DESCRIPTION OF THE DRAWINGS

The present invention is best understood when read in conjunction with the accompanying figures which serve to illustrate the preferred embodiments. It is understood, however, that the invention is not limited to the specific embodiments disclosed in the figures.

FIG. 1: Flow analysis of ES cells following staining with JC-1. X-axis; FL1 (green fluorescence, log scale), Y-axis; FL2 (red fluorescence, log scale). A) ES cells were cultured either in the presence or absence of LIF for 4 days. The numbers indicate the percentage of cells in the quadrants. B) Bar graph depicts the percentage of JC-1 red staining cells in culture after 4 days in either high glucose, low glucose, or galactose. Data for both a 129-derived ES cell line and C57B1/6 derived ES cell line are shown.

FIG. 2: Flow analysis and sorting of ES cells stained with the fluorescent dye, JC-1, which monitors mitochondrial inner membrane potential, ES cells were cultured in the presence of LIF for 2 days prior to sorting and stained with JC-1. The gates selected for sorting are shown. A) Pre-sort. B) Staining profiles of the two populations immediately following the sort. C) Sorted cell populations reanalyzed after 4 days in culture grown in the presence or absence of LIF.

FIG. 3: Offspring/chimeric frequency from ES cells following drug treatment.

FIG. 4: Assessment of the extent of ES cell contribution in chimeras (i.e., in which the agouti coat contribution was equal to or exceeded 60% of the total coat color); JC-1 green cells yield approximately 6 times as many highly chimeric mice as JC-1 red/green fluorescing cells.

### DETAILED DESCRIPTION OF THE INVENTION

c) Definitions

The following definitions are set forth to illustrate and define the meaning and scope of the various terms used to describe the invention.

The term “stem cell” means an unspecialized cell that is capable of replication or self-renewal. A stem cell can develop into specialized cells of a variety of cell types. The stem cell is also known as a cell that, upon division, produces dissimilar daughters, wherein one daughter cell is replacing the original stem cell, and the other daughter cell is differentiating further.

The term “embryonic stem (ES) cells” refers to stem cells taken from human embryos. Human embryonic stem cells are self-renewing cells that are derived from in vitro fertilized blastocysts.

“Hematopoietic stem cells (HSC)” are cells that give rise to distinct daughter cells, wherein one daughter cell is a replica of the stem cell, while the other daughter cell is a cell that will further proliferate and differentiate into a mature blood cell. HSC are found in blood (adult and umbilical cord) and bone marrow.

By the term “unipotent stem cell” is meant, for the purpose of the specification and claims, a stem cell that divides as well as gives rise to a single mature cell type (e.g., a spermatogenic stem cell).

By the term “pluripotent stem cell” is meant, for the purpose of the specification and claims, a stem cell that includes in its progeny all cell types that can be found in a postimplantation embryo, fetus, or developed organism.

“Somatic cells” are all cells of the body other than egg or sperm cells. The term “somatic stem cell” or “adult stem cell”, as used herein, means an undifferentiated cell found in a differentiated tissue that can renew itself and differentiate to yield all the specialized cell types of the tissue from which it originated.

The term “preimplantation embryo” means a very early, free-floating embryo, from the time the egg is fertilized until implantation in the mother's womb is complete.

A “primordial germ cell” means a stem cell that has started differentiating down the path of a germ cell, but has not yet developed into a germ cell. A germ cell refers to a sperm or an egg cell.

A “blastocyst” refers to the developmental stage of a fertilized ovum when it is ready to be implanted. The blastocyst includes an inner cell mass and an internal cavity. The outer layer of cells is called the trophoblast. A fertilized egg (zygote) becomes a blastocyst before differentiation into three germ layers.

By the term “chimera” is meant, an organism that is comprised of cells from two or more zygotes, or cells from a zygote and embryonic stem (ES) cells. A zygote is a mostly diploid cell that is formed by the union of two gametes or reproductive cells (i.e., an ovum or female gamete fertilized by a sperm or male gamete). A “chimeric animal” refers to an animal that is derived from the fusion of two or more preimplantation embryos or an embryo and ES cells.

d) Respiratory Function and Differentiation of ES Cells

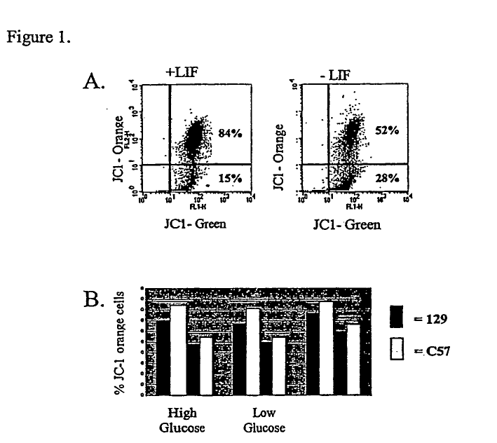

Measurements of the mitochondrial membrane potential in ES cells revealed sub-populations of cells that are sustained by oxidative phosphorylation or by glycolysis. In one embodiment, JC-1 dependent fluorescence was used to monitor mitochondrial oxidative phosphorylation. The mitochondria specific fluorescent dye, JC-1, exists as a monomer at low inner-membrane potential (<100 mV), emitting in the green range, but forms “J-aggregates” at higher potentials (>140 mV) which changes the emission into the red range. In a preferred embodiment, JC-1 fluorescence is used to measure respiratory activity in live cells. In order to assess if cells are primarily using oxidative phosphorylation or glycolysis, mitochondrial inner-membrane potential can be assayed by JC-1 fluorescence (1 ug/ml) in ES cells grown in both the presence (undifferentiated) and absence (differentiation) of LIF. Under both culture conditions two sub-populations of ES cells, fluorescing either green (glycolysis) or red (oxidative phosphorylation) are detected. The number of cells using oxidative phosphorylation increases when the cells are grown undifferentiated in LIF (FIG. 1A).

Selection of ES cells for oxidative phosphorylation shows a sub-population of cells surviving on glycolysis. If grown in galactose as the sole carbon source, a sub-population of cells need to engage in active oxidative phosphorylation to convert galactose to glucose. In one embodiment, ES cells are selected in galactose-containing culture medium for an incubation period of about four days. Under these conditions ES cells increase their doubling time by about 25% from 16 to 20 hours, and the acidity of the medium is greatly reduced over standard conditions. Following JC-1 staining and flow analysis, the sub-population of ES cells showing JC-1 red fluorescence is slightly increased (FIG. 1B). However, JC-1 staining indicates that there may still be a population of cells that does not use oxidative phosphorylation.

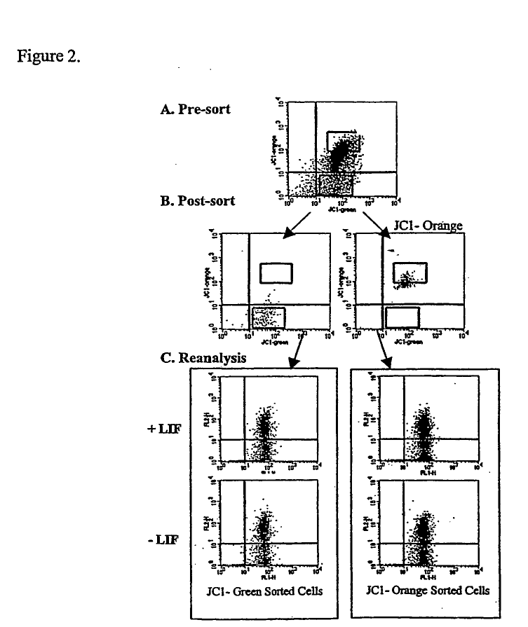

Assays with flow-sorted ES cells show retention of the ability to expand in vitro and to differentiate by generating mouse chimeras. In one embodiment, JC-1 staining is not toxic to ES cells. In another embodiment, the differences between the sorted sub-populations in their abilities to generate mouse chimeras are discussed herein (vide infra). When ES cells are stained, flow sorted (FIGS. 2A and 2B), and expanded for 96 hours (i.e., the different sub-populations), the cells grow at about the same doubling time (˜16 hours) as before staining and sorting. Following re-staining and FACS analysis, ES cells re-establish the original sub-populations corresponding to distinct respiratory activities (FIG. 2C). Thus, no overt JC-1 mediated toxicity exists. For chimera studies, ES cells may be injected into blastocysts and transferred to recipient foster mothers using methods known to those skilled in the art. Offspring can then be evaluated for ES cell-derived coat pigmentation (agouti) vs. the host strain coat (black). Chimeras generated from JC-1-green cells are more than two times as abundant, 48% vs. 21%, than those from JC-1-red cells (Table 1). In addition, chimeras displaying agouti pigmentation in more than 60% of the coat are more than three times as frequent from JC-1 green cells than from JC-1 red cells (26% for JC-1-green, vs. 8% for JC-1-red; see Table 1 below).

TABLE 1
Chimera Analysis Following Injections of FAC
Sorted JC-1 Green and JC-1 Red ES Cells

| Totals | JC-1 Red | JC-1 Green |
|---|---|---|
| Chimeras | 5 | 11 |
| Non-Chimeras | 19 | 12 |
| Chimeras/Total Mice | 21% | 48% |
| % Chimerism | | |
| 0-60% | 3 | 5 |
| 60-100% | 2 | 6 |
| >60% chimeric | 8% | 26% |

It is an object of this invention to show that ES cell cultures consist of subpopulations of cells which differ in their ability to differentiate. In order to establish the relationship between ES cell JC-1 fluorescence, respiration and differentiation permissiveness, cells may be grown under hypoxic (<2% oxygen) or normnoxic conditions and assayed for JC-1 red/green ratio, oxygen consumption and lactate secretion, proliferation rate, ES cell self-renewal (STAT3 activity), and differentiation permissiveness by chimeric assays. ES cells with high mitochondrial membrane potential (as shown by high JC-1 red/green ratio) display low differentiation permissiveness, while those with low mitochondrial membrane potential (JC-1 green) display high differentiation permissiveness. Control normoxic cells exhibit a JC-1 red/green ratio of about 16:1; those grown in galactose rich medium exhibit a ratio of about 30:1; and cells treated with FCCP (20) to reduce membrane potential exhibit a ratio of about 6:1. Measurements of lactate secretion after 24 hours can be surprisingly similar for both normoxic and hypoxic ES cells (about 80 mg/dl), while, as expected, the galactose-treated cells produce relatively little lactate (<10 mg/dl), which is indicative of commitment to the OXPHOS metabolic pathway. ES cells on galactose also consume about 50% more oxygen than those sustained on glucose. ES cell proliferation rates for control and hypoxic cells (both with and without LIF) may be similar. The addition of 2-deoxy glucose (0.5 mM), oligomycin (3 μM) or FCCP+ oligomycin usually dramatically reduces proliferation rates, by as much as 3-fold for the 2-dG. STAT3 activity may be similar for normoxic and hypoxic cells. Interestingly, annexin V-positive gated live cells may not show green JC-1 staining, while gated necrotic cells may be predominantly red stained, contrary to the conventional belief that green cell staining indicates apoptosis.

e) Subpopulations of JC-1 Stained Cells

In one aspect of the invention, subpopulations of embryonic stem (ES) cells are identified using the fluorescent dye, JC-1, combined with FACS analysis. These subpopulations are evaluated as to their capacity to differentiate by injection into blastocysts. Furthermore, they differ in their ability to produce chimeric mice (see Table 2, vide infra). The JC-1 green subpopulation may be eliminated using inhibitors for multidrug resistance (MDR)-related gene products, including but not limited to, verapamil, cyclosporin A, and reserpine. Several MDR gene family members are assessed for expression by RT-PCR. In one embodiment of the invention, the levels of transcript of the mdr-1a, mdr-1b, and mrp-1 genes (i.e., genes coding for ABC transporters) are consistently higher in the JC-1 drug-sensitive population. Thus, these ABC transporters may be involved in defining the subpopulation that shows increased ability for differentiation.

In a preferred embodiment, the cationic dye JC-1 is used as a probe to explore whether or not heterogeneities among ES cells in culture may be present. The probe allows for monitoring of mitochondrial activity. JC-1 is a positively charged lipophilic molecule that accumulates and forms aggregates at the charged mitochondrial inner-membrane (Cossarizza et al. (1996) Exp. Cell Res. 222:84-94). The dye's fluorescence properties are employed to perform ratiometric analysis (JC-1 red/JC-1 green) of cells following exposure to physiological and pathological conditions. The dye also allows for the analysis of the ratio of charged to uncharged mitochondria in a single cell (Wilding et al. (2001) Hum. Reprod. 16:909-917). When using JC-1, similar as with most indicator dyes, the fluorescence signal is dependent on the concentration of the dye within the cell, which can be affected by mechanisms unrelated to the membrane potential. Such mechanisms may include efflux pumps that can extrude a large number of different compounds. The most prominent of these pumps is P-gp, encoded by MDR-1. Members of the MDR gene family are sensitive to specific inhibitors, such as verapamil, cyclosporin A, and reserpine. The exposure of ES cells to these inhibitors eliminates almost the entire JC-1 green subpopulation, suggesting that this population is due to MDR mediated dye efflux. However, these inhibitors also affect mitochondrial Ca2+ concentrations, thereby accounting for changes in JC-1 fluorescence due to mitochondrial permeability transition and ψm.

In one aspect of the invention, staining patterns of the dyes JC-1 and Hoechst 33342 are compared. Hoechst 33342 is a dye that localizes to the nucleus and is well characterized as to its sensitivity to MDR inhibitors in hematopoietic stem cells (HSC) (Goodell et al. (1996) J. Exp. Med. 183:1797-1806); Scharenberg et al. (2002) Blood 99:507-512; Zhou et al. (2001) Nat. Med. 7:1028-1034). Similar to the JC-1 green subpopulation, the Hoechst side population (SP) can be eliminated by MDR inhibitors, suggesting that loss of the JC-1 green population is predominantly due to dye efflux activity. In one embodiment of the invention, specific gene activities that are responsible for JC-1 dye extrusion in ES cells are evaluated by using RT-PCR, particularly the expression levels for a number of MDR gene family members. Consequently, mdr-1a, mdr-1b, and mrp-1 are found to be expressed at higher levels in JC-1 green cells than in JC-1 red cells, suggesting that several MDR activities account for at least some of the dye efflux (see Example 17, vide infra). Expression of MDR gene family members is known to be important for stem cell populations from various lineages, including the hematopoietic and muscle lineages (Bunting, K. D. (2002) Stem Cells 20:11-20). In the hematopoietic lineage, MDR and related activities define the Hoechst SP, which is associated with a long term reconstituting activity (Goodell et aL (1996) (supra). In addition, in HSC, the major Hoechst efflux activity is due to expression of the breast-cancer-resistance-protein, bcrp1 (Thou et al. (2001) (supra).

The physiological role of increased ABC transporter expression in ES cells and other stem cells may be the removal of toxic metabolites. This includes reactive oxygen species (ROS) generated during respiration. For example, an increased demand for removal of ROS may become necessary as part of the switch from OXPHOS to glycolysis, requiring increased levels of MDR-1 and possibly other members of the family of ABC transporters. In fact, a link between hypoxia and MDR-1 expression does exist (Comerford et al. (2002) Cancer Res. 62:3387-3394). Hypoxia causes stabilization of hypoxia-inducible-factor-1α (HIF-1α), a transcription factor that is expressed constitutively, but rapidly degraded under ambient oxygen concentration (Carmeliet et al. (1998) Nature 394:485-490; Seagroves et al. (2001) Mol. Cell Biol. 21:3436-3444). Stabilized HIF-1 alpha leads to activation of hypoxia response genes such as vascular endothelial growth factor (VEGF), the inducible form of nitric oxide synthase (iNOS), and enzymes regulating glycolysis, such as phosphoglycerate kinase (PGK), among others. Hence, glycolytic ES cells may be subject to HIF-1α dependent up-regulation of mdr-1 gene expression.

Embryonic stem cells make it possible to derive permanent cultures from human embryos. Because of the pluripotent nature of these cells, novel clinical strategies for stem cells concerning cell and tissue regeneration are potentially valuable. However, critical shortcomings in the understanding of stem cells still exist in the art. Thus, it is an object of this invention, to remedy some of these shortcomings. For example, the invention provides functionally distinct ES cells that differ in their ability to differentiate in vivo, which is a paramount step toward that goal. In addition, the identification and separation of subpopulations of ES cells greatly improves the efficiency of differentiation in vitro. In one embodiment of the invention, the underlying molecular mechanisms defining these subpopulations are employed to manipulate the cultures and shift cells between subpopulations.

In another aspect of the invention, ES cells can be continuously cultured and maintained in an undifferentiated state (i.e., indefinitely), as such, they present the most appropriate system to understand processes such as immortality. Some ABC transporters may play key roles in determining processes such as life span.

f) Examples

The following specific examples are intended to illustrate the invention and should not be construed as limiting the scope of the claims.

### EXAMPLE 1

### First ES Cell Culture

Mouse embryonic stem (ES) cells were cultured under standard conditions, which includes the use of fibroblast feeder cells. Feeder cells were derived from mouse fetuses at day 13, expanded for 3 passages and mitotically inactivated by irradiation with gamma rays to produce mouse embryonic fibroblasts (MEF). Embryonic stem cell culture media consisted of Dulbecco's modified Eagle's medium (DMEM), supplemented with 15% fetal calf serum, non-essential amino acids, beta mercaptoethanol, penicillin-streptomycin and leukemia inhibitory factor (500 units/ml). ES cells were either cultured in 6-well dishes or in 10 cm dishes. At splitting, ES cells were seeded at a density of 5×10⁴ cells/cm² and cultured for 48 hours, until furtherpassaged at a ratio of 1:8 to 1:10. The acidity of the culture media was controlled by daily media changes.

### EXAMPLE 2

### FACS Analysis and Mitochondrial Staining

The cells of Example 1 were stained with 1 μg/ml 5,5′,6,6′-tetrachloro-1,1′,3,3′-tetraethyl-benzimidazolylcarbocyanine iodide (JC-1, Molecular Probes, Eugene, Oreg.) and flow cytometry was performed using a FACSCalibur flow cytometer (Becton Dickinson, San Jose, Calif.). Cells were stained as above and sorted using a FACSVantage SE cell sorter (Becton Dickinson).

### EXAMPLE 3

### Blastocyst Injection

Sorted ES cells were dislocated and dispersed into a single cell suspension using trypsin. Trypsinization was performed as follows: Culture medium in ES cell dishes was aspirated and the cells are washed once with calcium/magnesium-free phosphate buffered saline (PBS). After the PBS was aspirated, 1 ml of trypsin EDTA was added to a 10 cm plate and the cells incubated at 37° C. for 9 to 12 minutes. Subsequently, the trypsin was quenched with medium containing serum and tightly associated cell clusters dispersed by pipeting gently several times. Single cell suspensions were then centrifuged at 80 g for 5 minutes, the supernatant aspirated, and the cell pellet re-suspended in injection medium, consisting of Opti-MEM (Hepes-buffered) supplemented with 10% FCS, and Pen-Strep. In this medium, cells were chilled to around 8 to 10° C. prior to transfer into the injection chamber.

### EXAMPLE 4

### Production of Mouse Blastocyst Stage Embryos

Female mice from strain C57BL/6, age 6 to 12 weeks of age, were hormone primed using the following regimen. Follicle stimulating hormone in the form of pregnant mare serum (PMS), 0.5 IU/mouse (0.1 ml) was administered by injection intraperitoneally 46 hours prior to mating. Forty six to forty eight hours later, human chorionic gonadotropin, hCG (0.5 IU/ml) was administered intraperitoneally and the female mice mated by placement in the cages overnight with C57BL/6 male mice. The following day, females were checked for successful mating by the presence of a copulation plug. The day following the night of mating is counted as day 1 in embryo development. Blastocysts were harvested on day 4 by sacrificing the females by cervical dislocation. Both uterine horns were removed and carefully cleaned from tissues and blood vessel lining the uterine horns. Following clean up of the tissues, a 1 cc syringe with a 25 gauge needle fitted were filled with DMEM, 10% FCS and Penstrep in order to flush the embryos from the cavity of the uterine horns.

### EXAMPLE 5

### Injection of Mouse Blastocysts with Embryonic Stem Cells

ES cells were injected into blastocysts using an inverted microscope equipped with Nomarski optics and micromanipulators. As injections of ES cells are performed at 8 to 10° C., the stage is further equipped with a cooling device which is based on the Peltier principle. For injections, ES cells and blastocyst stage embryos were placed in medium which is specifically prepared for injections containing DMEM buffered with 20 mM HEPES supplemented with Pen-strep. A drop of this medium was placed in a slide consisting of a glass cover slip mounted to an aluminum frame for adequate heat conduction. Once the cells and embryos were placed in the medium and mounted on the microscope stage, the injection needle and the holding pipette which are mounted to the micromanipulator holding devices were adjusted to the proper positions on the stage. Using the injection needle connected to a micrometer syringe, ES cells were then aspirated individually, approximately 100 to 150 cells at a time, followed by the holding pipette picking up a blastocyst by force of suction. It was positioned such that the inner cell mass is located adjacent to the end of the holding pipette, with the area of the single layer of the trophectoderm next to the tip of the injection needle. Upon focusing at an intercellular junction in the trophectoderm layer, the needle tip was pushed through the zona pellucida and the trophectoderm. As the needle tip is located in the blastocoel cavity, positive pressure was applied gently to expel ES cells into the blastocyst. Around 12 to 16 cells were injected per embryo, which then were placed into the 37° C. incubator for recovery from the injection.

### EXAMPLE 6

### Embryo Transfers

Injected embryos were transferred into pseudopregnant recipient foster mother mice by uterine transfer. Foster mother mice had previously been mated with vasectomized males two and a half days prior to the surgical embryo transfer and were first prepared for surgery by total anesthesia. Following anesthesia, they were placed in a stage of a dissecting scope, an incision made in the lumbar region of the left back and the left uterine horn exteriorized. Using a 30 gauge needle, a small whole was made in the uterine horn proximal to the oviduct. Subsequently, 8 to 10 embryos were transferred using a transfer pipette attached to a mouthpiece to apply air pressure.

### EXAMPLE 7

### Treatment of Offspring and Animal Husbandry

Term development of embryos is completed 17 days following injection. Around 8 to 10 day post partum, the coat becomes distinctly pigmented such that it is possible to discern between black and the agouti color, which is characterized by the yellow band in the black bristles giving a brown appearance. At weaning, which is reached at 3 weeks of age, offspring animals were separated from their mothers and either genotyped or housed until maturity and mating to check for germline transmission of the ES cell genotype.

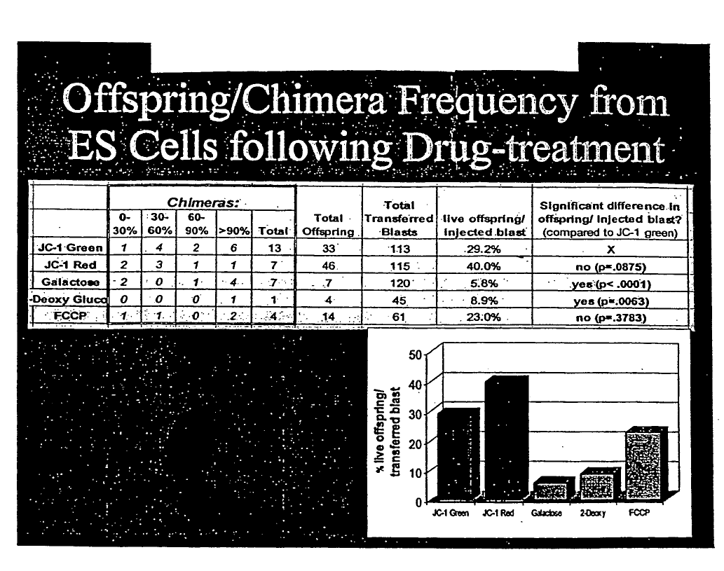

Additional studies were conducted using ES cells treated with galactose, 2-deoxy glucose or FCCP. The results are summarized in FIG. 3.

### EXAMPLE 8

### Second ES Cell Culture

ES cells were maintained in ES cell media consisting of DMEM (no pyruvate, high glucose) (Gibco-BRL), 15% fetal calf serum (FCS) (Hyclone), and supplements (as described in Robertson, E. J. (1987) IRL Press Limited, Oxford, page 71-112). Supplemental Leukemia Inhibitory Factor (LIF) (1:1000 dilution) was prepared by transfecting COS-7 cells (ATCC) with a LIF expression vector, pCAGGS-LIF, then collecting and testing the supernatant 48 hours later (as described in Smith, A. (1991) Journal of Tissue Culture Methods 13:89-94). Irradiated mouse embryonic fibroblasts (MEFs) were derived from d13 embryos for use as feeder cells (as described in Robertson (1987) (supra)). MEFs were plated at a concentration of 105/cm², 1-5 days prior to plating of ES cells. ES cells were passaged once onto gelatin-coated TC plates prior to plating at 2×105 cells/ml on gelatin-coated 24 well plates for FACS assays. Cell cultures were allowed to incubate 24-48 hours in standard ES cell media prior to trypsinization and various treatment conditions. For MDR inhibitor assays, verapamil (Sigma) was used at 100 μM. Reserpine (Sigma) and Cyclosporin A (Sigma) were used at various concentrations and added to cells 30 minutes prior to FACS staining as described below. The Jurkat clone E6.1 (ATCCT1B-152) was used as a negative control for MDR activity (Labroille et al. (2000) Cytometry 39:195-202). Cells were maintained in RPMI-1640+10% FBS with 2 mM L-glutamine, 1.5 g/L sodium bicarbonate, 4.5 g/L glucose, 10 mM HEPES, and 1.0 mM sodium pyruvate.

### EXAMPLE 9

### Chimera Construction

Germline-competent ES cells were microinjected into d3.5 blastocysts harvested from 4-6 week old C57B1/6 female mice according to the protocol described by Bradley et al. (Bradley and Robertson (1986) Curr. Top. Dev. Biol. 20:357-371). Approximately 12-15 cells were injected into each blast. 8-10 injected blasts were transferred into the uteri of pseudo-pregnant B6D2F1 mice (Taconic). Resulting offspring were assessed by the degree to which the ES cells (agouti) contributed to coat color over native blastocyst cells (black).

### EXAMPLE 10

### FACS Analysis of ES Cells Showing Differences in Multidrug Resistance Activity

For MDR inhibitor dose response assays, cells were trypsinized for 15 minutes, quenched with ES cell media, triturated to a single cell suspension, and centrifuged at 1000 rpm for 5 minutes. Cells were resuspended at a concentration of 1×106/ml in ES cell media with LIF in the presence or absence of MDR inhibitors. Following a 30 minute incubation, 100 μl of a 10× solution containing 10 μg/ml 5,5′,6,6′-tetrachloro-1,1′,3,3′-tetraethyl-benzimidazolyl-carbocyanine iodide (JC-1) (Molecular Probes, Eugene, Oreg.) were added, and the cells were incubated for 30 minutes at 37° C. Following centrifugation, cells were washed 1×0 with cold HBSS, centrifuged and resuspended in cold PBS at the same concentration, placed on ice, and analyzed within 60 minutes (as described in Cossarizza et al. (1993) Biochem. Biophys. Res. Commun. 197:40-45). Flow cytometry was performed using a FACSCalibur flow cytometer (Becton Dickinson, San Jose, Calif.). For sorting, cells were stained as above, resuspended in cold Opti-MEM (Gibco-BRL)+1% FCS at 1×106 cells/ml and sorted using a FACSVantage SE cell sorter (Becton-Dickinson). Following cell sorting, they were injected into d3.5 blastocysts from mouse strain C57BL/6, and uterine transfers were performed according to published standard procedures (Hogan et al. (1994) Manipulating the Mouse Embryo: A Laboratory Manual: Cold Spring Harbor Laboratory Press; Robertson (1987) (supra)). Offspring were assessed as to the percentage of agouti coat color they exhibited.

### EXAMPLE 11

### Hoechst 33342 Side Population Assay

ES cells were trypsinized as above, centrifuged and resuspended at 1×106 cells/ml in ES cell media in the presence or absence of 100 μM verapamil, and incubated for 30 minutes. Following the incubation, 100 μl of 50 μg/ml Hoechst 33342 (Molecular Probes, Eugene, Oreg.) were added per ml of cells for a final concentration of 5 μg/ml and incubated for 90 minutes at 37° C. (as described in Goodell et aL (1996) J. Exp. Med. 183:1797-1806). For double staining, JC-1 was added as a 10 × solution (100 μl of a 10 μg/ml concentration) during the last 30 minutes of incubation. Following centrifugation, cells were washed 1× with cold HBSS, centrifuged and resuspended in cold PBS at the same concentration, placed on ice and analyzed within 60 minutes. Flow cytometric analysis was performed using a Becton Dickinson (San Jose, Calif.) FACS Vantage flow cytometer configured for dual-emission wavelength analysis (as described in Goodell et al. (1996) (supra)).

### EXAMPLE 12

### Analysis of Gene Expression

Total RNA was harvested from JC-1 stained cells that were FACS-sorted for high or low red fluorescence by use of the RNeasy Mini Kit (Qiagen). RNA was quantitated using the RiboGreen RNA Quantitation kit (Molecular Probes), and 2 μg of RNA was used to synthesize complementary DNA (cDNA) using the ThermoScript RT-PCR System (Invitrogen) with 50 ng of random hexamer primers. The RT reactions were then subjected to real-time PCR analysis for quantitation of the β-actin signal in each sample. The β-actin primer sequences were: Forward 5′-CCTAAGGCCAACCGTGAAAA-3′ Reverse 5′-GAGGCATACAGGGACAGCACA-3′. All RT reactions were normalized to B-actin to obtain equivalent B-actin signals for a given cycle number. Semiquantitative RT-PCR of mdr-1a, mdr-1b, mdr-2, mrp-1, mrp-2, and β-actin transcripts in sorted cell populations was performed by amplification from 1:2 dilutions of the normalized RT reactions using Platinum Taq DNA Polymerase (Invitrogen). Gene-specific primer sequences for mdr-1a, mdr-1b, mdr-2 and mrp-1 murine cDNAs (Zhou et al. (2001) Nat. Med. 7:1028-1034) are as follows:

| | | |
|---|---|---|
| mdr-1a: | Forward | 5′-AGCTGGAGAGATCCTCACC-3′, |
| | Reverse | 5′-CTGTAGCTGTCAATCTCGGG-3′ |
| mdr-1b: | Forward | 5′-AGCCGGAGAGATCCTCACC-3′ |
| | Reverse | 5′-CTGTAGCTGTCAATCTCAGG-3′ |
| mdr-2: | Forward | 5′-AGCTGGAGAGATCCTCACC-3′ |
| | Reverse | 5′-CTGTAGCTGTCAATCAGAGG-3′ |
| mrp-1: | Forward | 5′-GGCGCTGTCTATCGTAAGGC-3′ |
| | Reverse | 5′-GACCTCCGCTCAATGCTGT-3′ |

Primer sequences for mdr-2 are as follows (Yu et al. (2002) Life Sci. 70:2535-2545):

| | |
|---|---|
| Forward | 5′-TGCCTGTCCTATAACTCACGGATT-3′ |
| Reverse | 5′-AGCAAATGTTATTGTTTGTAGGTCCG-3′ |

Amplification was performed over 30 cycles of 94° C. for 30 seconds, 60° C. for 1 minute, and 72° C. for 30 seconds using a Perkin-Elmer Thermocycler. The PCR products were electrophoresed on a 3% NuSieve agarose gel. The gel was stained with ethidium bromide and photographed.

### EXAMPLE 13

### JC-1 Subpopulations of ES Cells

In order to evaluate the functional heterogeneity of ES cells, the cationic dye, JC-1, was used. JC-1 senses the mitochondrial inner membrane potential, ψm. The fluorescence emission wavelengths for JC-1 depend on the concentration it reaches at negatively charged membranes. High dye concentration, which is charge dependent, results in aggregate formation and exhibits red fluorescence (590 nm). This concentration normally only occurs in mitochondria when. ψm>140 mV. At a low concentration JC-1 monomers fluoresce green (530 nm). Staining of ES cells in suspension followed by flow cytometrical analysis using two channel analysis revealed that the majority of cells fluoresce both red and green. However, a subpopulation of cells only showed green fluorescence. These cells were indistinguishable from the red/green fluorescing cells by morphological criteria, as well as in forward and side scatter by flow cytometry and confocal microscopy. As a control experiment JC-1 staining was evaluated in a Jurkat T cell line. The resulting flow cytometry analysis showed that control cells are almost uniformly red/green. Hence, these data established that JC-1 fluorescence identifies two subpopulations of ES cells.

### EXAMPLE 14

### Dynamic Staining Pattern of JC-1 Subpopulations of ES Cells

Subsequently, two further objectives were investigated. The first objective was to assess if JC-1 was toxic to ES cells. The second objective was to check if sorted subpopulations were static and/or clonal. In order to address the first objective, ES cells were stained and sorted for “JC-1 red/green” and “JC-1 green only” subpopulations using FACS analysis. Both sorted subpopulations grew at approximately the same doubling time (12 to 16 hours) as original unstained cultures, suggesting that no overt JC-1 mediated toxicity could be detected. The second objective was addressed by re-staining sorted and subcultured ES cells with JC-1 followed by FACS analysis. The original JC-1 staining profile was re-established after expansion of sorted subpopulations regardless of whether cells originated from red/green or green-sorted cells.

### EXAMPLE 15

### JC-1 Green Fluorescing ES Cells with Increased Ability to Produce Chimeric Mice

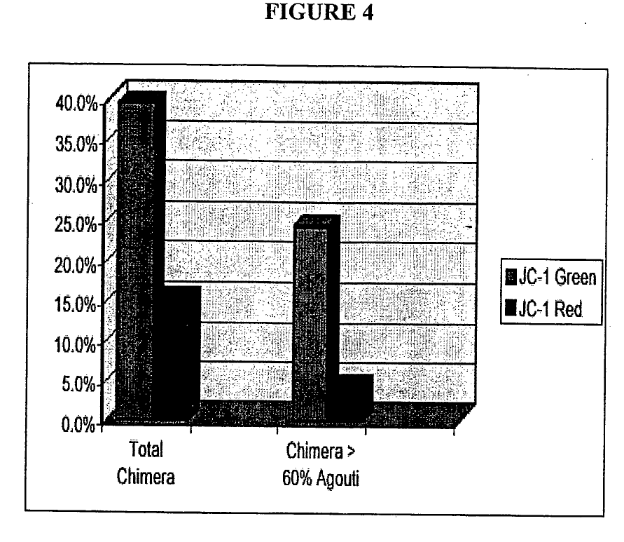

Following initial identification of the subpopulations and assessment of their dynamic staining pattern, their potential to differentiate was investigated through chimera analysis. In order to assess this potential, cells were again subjected to FACS following staining with JC-1. Sorted subpopulations were injected into blastocyst stage embryos according to a standard protocol used to generate chimeras (Stewart, C. L. (1993) Methods Enzymol. 225:823-855). As shown in Table 2 below, offspring from JC-1 red/green and JC-1 green fluorescing cells were scored as to the degree of agouti coat color (129SvEv-ES cell contribution) versus black coat color (C57/BL6-derived blastocysts). Data from 8 different experiments revealed that the number of chimeras born from JC-1 green cells was significantly higher than from JC-1 red/green cells (Student's T test: p=0.0035). In fact, ES cells fluorescing JC-1 green yielded twice as many chimeras as cells fluorescing red/green. Also significantly different was the degree of chimerism. An assessment of the extent of ES cell contribution in chimeras (i.e., in which the agouti coat contribution was equal to or exceeded 60% of the total coat color) revealed that JC-1 green cells yielded approximately 6 times as many highly chimeric mice as JC-1 red/green fluorescing cells. A graphic representation of the data in Table 2 is shown in FIG. 4.

TABLE 2

| | Chimeras | | | # Blasts | Total |
|---|---|---|---|---|---|
| | 0-60% | 60-100% | Total | injected | Offspring |
| JC-1 Green | 5 | 8 | 13 | 113 | 33 |
| JC-1 Red | 5 | 2 | 7 | 115 | 46 |

### EXAMPLE 16

### JC-1 Fluorescence Sensitivity to MDR Inhibitors

Using the cationic fluorescent dye, JC-1, functionally distinct populations of ES cells were identified. An objective of this invention was to shed some light onto the underlying molecular mechanisms accounting for ES cell heterogeneity. A possible mechanism may involve MDR mediated efflux of JC-1. Hence, specific MDR inhibitors were investigated, including verapamil, cyclosporin A (CsA), and reserpine. Specifically, cells were prepared under standard conditions, in the presence and absence of these inhibitors, followed by staining with JC-1 and flow cytometry. The addition of inhibitors CsA (20 μM), verapamil (200 μM), and reserpine (10 μM) resulted in a dramatic shift of the majority of cells towards red fluorescence. Generally, less than 5% of the cells maintained a “JC-1 green only” fluorescence profile when treated with any of these inhibitors. The JC-1 green population may be due to increased dye efflux activity in ES cells. This conclusion was further supported by data showing that the effect of these inhibitors is dose dependent. In fact, saturation inhibition can be achieved with all three inhibitors at their respective concentrations. CsA appeared to be the most effective as a 20 μM dose reduced the JC-1 green population to approximately 2% of the total number of cells, with a minimal mean error.

### EXAMPLE 17

### Multidrug Resistance and ABC Transporter Gene Expression in ES Cell Subpopulations

Another objective of the invention concerned the identification of specific molecular mechanisms responsible for dye efflux. As the MDR inhibitors showed broad substrate specificity, candidate MDR genes and related genes that belong to this family of ABC transporters were investigated. Gene expression levels were assessed using semi-quantitative reverse-transcriptase polymerase chain reaction (RT-PCR) analysis. This analysis included the following MDR family members: mdr-1a, mdr-1b, mdr-2, mrp-1, and mrp-2. In order to calibrate each batch of cDNA prepared from quantitated RNA obtained from sorted ES cells, real-time PCR analysis for beta-actin cDNA was performed. Upon normalization of RNA samples, RT-PCR analysis was performed for the genes listed above. The signals for mdr-1a and mdr-1b, as well as mrp-1 but not mrp-2, were detected at higher levels in the JC-1 green subpopulation. This strongly suggested that these transporters are involved in JC-1 dye efflux in ES cells.

### EXAMPLE 18

### Comparison of Staining Profiles between JC-1 and Hoechst 33342 in ES Cells

In pluripotent HSC (hematopoietic stem cells), Hoechst 33342 (Hoechst) (i.e., a stain that has been used extensively for the phenotypic characterization of stem cells) defines a characteristic low fluorescence-intensity side population (SP) of cells. This SP is a consequence of expression of specific drug efflux activities including members of the MDR gene family and another ABC transporter, bcrp/ABCG2 (Scharenberg et al. (2002) Blood 99:507-512; Zhou et al. (2002) Proc. Natl. Acad. Sci. USA 99:12339-12344; Zhou et al. (2001) Nat. Med. 7:1028-1034). HSC that fall within the Hoechst SP have the greatest potential to reconstitute all lineages long term (Goodell et al. (1996) J. Exp. Med. 183:1797-1806). Similar to HSC, ES cells show a characteristic Hoechst SP, which may suggest that these cells have an increased ability to produce chimeras in comparison to unsorted ES cells (Bunting, K. D. (2002) Stem Cells 20:11-20; Zhou et al. (2001) (supra)). Hence, another objective of this invention was to assess whether ES cells belonging to the JC-1 green subpopulation were identical to the Hoechst SP or if there were detectable differences. Double staining of ES cells using both JC-1 and Hoechst followed by four channel analysis (Hoechst: red [675 nm] and blue [450 nm]; and JC-1: red [590 nm] and green [530 nm]). The analysis indicated that JC-1 staining was somewhat compressed, while the Hoechst staining showed the characteristic side population. In order to determine if the JC-1 green subpopulation was identical to the Hoechst SP, the degree of overlap between the two populations was evaluated. Both, JC-1 green and JC-1 red cells were found within the Hoechst SP. Reciprocal analysis, using the JC-1 green-gated population, showed that almost all of these cells defined a characteristic Hoechst linear pattern, indicating that these two populations, Hoechst SP and JC-1 green, are not identical. The four-color analysis suggested that JC-1 delineates a further subset of cells within the Hoechst side population.

Various modifications and variations of the present invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the claims. All publications, patents, and other reference materials referred to herein are incorporated herein by reference.

What is claimed is:

1. An isolated stem cell sustainable in culture under glycolytic conditions and which maintains the potential to differentiate.

2. The stem cell of claim 1 which is unipotent or pluripotent.

3. The stem cell of claim 1 which is an embryonic or somatic stem cell.

4. The stem cell of claim 3 which is a pluripotent cell from a preimplantation embryo.

5. The stem cell of claim 1 which is a primordial germ cell.

6. The stem cell of claim 1 selected from the group consisting of hematopoietic, neuronal and mesenchymal stem cells.

7. An isolated stem cell which cell shows characteristic green staining with the mitochondrial marker JC-1.

8. An isolated stem cell which cell displays a low mitochondrial inner membrane potential based upon JC-1 green staining.

9. An isolated stem cell which cell displays a high mitochondrial inner membrane potential based upon JC-1 red staining.

10. A method of isolating a stem cell, comprising the steps of:
(a) isolating a blastocyst;
(b) identifying those cells which rely upon glycolysis for survival;
(c) isolating a glycolytic cell from the inner cell mass of the blastocyst; and
(d) culturing the isolated glycolytic cell to obtain an isolated stem cell.

11. The method of claim 10, wherein the cells are identified by staining with the mitochondrial marker JC-1.

12. The method of claim 10, further comprising maintaining the isolated cells on a fibroblast feeder layer to prevent differentiation.

13. A chimeric animal produced from a cell of claims 1 or 9.

14. A method of producing a chimeric animal comprising
(a) isolating a blastocyst;
(b) identifying those cells which rely upon glycolysis for survival;
(c) isolating the glycolytic cells from the inner cell mass of the blastocyst;
(d) transfecting a desired gene into the glycolytic cells;
(e) injecting the transfected cells into recipient blastocysts;
(f) implanting the transformed blastocysts into a host uterus; and
(g) nurturing the blastocysts to develop to term.

15. A method of producing glycolytic-dependent cells, comprising the steps of:
(a) culturing cells under hypoxic conditions;
(b) identifying those cells which rely upon glycolysis for survival;
(c) isolating the glycolytic cells from the culture; and
(d) culturing the isolated glycolytic cells.

16. A stem cell of claims 1 or 9 which is a mammalian stem cell.

17. A chimeric mammal produced from a stem cell of claim 16.

18. An isolated stem cell, wherein said stem cell can be identified by staining said cell with the fluorescent dye JC-1.

19. The isolated stem cell of claim 18, wherein said cell is sensitive to inhibitors of multidrug resistance (MDR) targets.

20. The isolated stem cell of claim 19, wherein said inhibitors are selected from the group consisting of verapamil, reserpine, and cyclosporine A.

21. The isolated stem cell of claim 19, wherein the multidrug resistance (MDR) target is an MDR-like dye efflux pump.

22. A method of identifying functionally distinct stem cells, comprising:
(a) staining the cells with the fluorescent dye JC-1;
(b) sorting the stained cells by fluorescence activated cell sorting (FACS);
(c) analyzing said functionally distinct stem cells by comparing their sensitivity to inhibitors of multidrug resistance (MDR) targets; and
(d) identifying a MDR-inhibitor sensitive JC-1 subpopulation of cells.

23. The MDR-inhibitor sensitive JC-1 subpopulation of claim 22, wherein said subpopulation has an increased differentiation permissiveness.

24. A method of switching embryonic stem cells between two subpopulations, comprising:
a) exposing a JC-1 green subpopulation to inhibitors of multidrug resistance genes; and
b) overexpressing recombinant multidrug resistance genes in a JC-1 red subpopulation.

25. The method of claim 24, wherein said inhibitors are selected from the group consisting of verapamil, reserpine and cyclosporine.

26. A method of changing a cell's ability to differentiate by switching the subpopulations of claim 24.

27. An embryonic stem cell which is differentiated by the method of claim 24.
